(19) United States
(12) Patent Application Publication
(10) Pub. No.: US 2023/0289960 A1
(43) Pub. Date: Sep. 14, 2023

(54) STORAGE MEDIUM, INFORMATION PROCESSING DEVICE, AND IMAGE DIAGNOSIS SUPPORT METHOD

(71) Applicant: Fujitsu Limited, Kawasaki-shi (JP)

(72) Inventors: Masaki TAKEUCHI, Kawasaki (JP); Yoshimasa MISHUKU, Yokohama (JP); Masataka UMEDA, Kawasaki (JP)

(73) Assignee: Fujitsu Limited, Kawasaki-shi (JP)

(21) Appl. No.: 18/152,974

(22) Filed: Jan. 11, 2023

(30) Foreign Application Priority Data
Mar. 11, 2022 (JP) 2022-038323

(51) Int. Cl. G06T 7/00; G16H 30/40

(52) U.S. Cl. CPC G06T 7/0012; G16H 30/40; G06T 2207/20081; G06T 2207/30096

(57) ABSTRACT

A storage medium storing an image diagnosis support program for causing a computer to execute process that includes inputting input images to a first model that outputs, according to input images obtained by imaging a subject under the plurality of imaging conditions, an estimation result of a disease name of the, and a degree of contribution to estimation of each of input images for each of the imaging conditions; selecting, among input images, an image imaged under an imaging condition for estimation selected based on the degree of contribution; inputting the image imaged under the imaging condition for estimation to a second model that outputs an estimation result of a lesion part in the image according to the input image; and outputting the estimation result of the lesion part specified based on an output result of the second model.

### CROSS-REFERENCE TO RELATED APPLICATION

This application is based upon and claims the benefit of priority of the prior Japanese Patent Application No. 2022-38323, filed on Mar. 11, 2022, the entire contents of which are incorporated herein by reference.

### FIELD

The embodiment discussed herein is related to a storage medium, an information processing device, and an image diagnosis support method.

### BACKGROUND

In recent years, to specify a lesion part of a patient, medical images of brain magnetic resonance imaging (MRI), computed tomography (CT) perfusion, or the like have been captured, and image interpretation diagnosis using deep learning has been performed for these medical images.

Furthermore, in capturing these medical images, by switching an imaging protocol (imaging conditions), parameters to be used for imaging are changed and patterns of body tissues (water, fat, bone, and the like) to be emphasized become different.

For example, in brain MRI, imaging protocols such as FLAIR, MRA, DWI, ADC, T1, T1 weighted, T2, and T2* are known. Then, even in a case of imaging the same site (for example, the head), by switching the imaging protocol, visibility of the lesion (pathological change) in the medical images changes.

Therefore, there are cases where the pathological change can be easily found and cases where the pathological change can be less easily found in the medical images according to a type of a disease, depending on the imaging protocol. Therefore, the imaging protocol used for image interpretation diagnosis differs depending on the disease. In the case where the lesion (pathological change) in the medical images captured with a specific imaging program can be less easily found, the imaging protocol may be said to be less effective for the image interpretation diagnosis, or the imaging protocol may be said to have a small contribution to the image interpretation diagnosis.

In a case of performing image interpretation diagnosis for the purpose of diagnosing only one type of disease, it is sufficient to select and use a specific imaging protocol, but there are many cases where the patient often has a plurality of diseases at the same time. Therefore, in the case of performing image interpretation diagnosis, it is not desirable to limit and use the specific imaging protocol for the medical images used for inference from the viewpoint of avoiding oversight of diseases.

Therefore, in the existing image interpretation diagnosis using deep learning, machine learning and inference are performed using all of image data captured with a plurality of imaging protocols.

Japanese Laid-open Patent Publication No. 2018-175343, Japanese Laid-open Patent Publication No. 2019-82881, U.S. patent Ser. No. 10/311,566, and Love, Askell & Siemund, Roger & Andsberg, Gunnar & Cronqvist, Mats & Holtas, Stig & Bjorkman-Burtscher, Isabella. (2011)., “Comprehensive CTEvaluation in Acute Ischemic Stroke: Impact on Diagnosis and Treatment Decisions.”, Stroke research and treatment. 2011. 726573. 10.4061/2011/726573 are disclosed as related art.

### SUMMARY

According to an aspect of the embodiments, a non-transitory computer-readable storage medium storing an image diagnosis support program for causing a computer to execute a process, the process includes inputting a plurality of input images to a first model that is generated by using training data in which a plurality of training images obtained by imaging a first subject under a plurality of imaging conditions is associated with a disease name of the first subject, the first model outputting, according to input of a plurality of input images obtained by imaging a second subject under the plurality of imaging conditions, an estimation result of a disease name of the second subject, and a degree of contribution to estimation of each of the plurality of input images for each of the imaging conditions; selecting, among the plurality of input images, an input image imaged under an imaging condition for estimation selected based on the degree of contribution; inputting the input image imaged under the imaging condition for estimation to a second model that outputs an estimation result of a lesion part in the input image according to the input of the input image; and outputting the estimation result of the lesion part specified based on an output result of the second model.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a diagram illustrating a hardware configuration of an image interpretation diagnosis support system as an example of an embodiment;

FIG. 2 is a diagram illustrating a functional configuration of the image interpretation diagnosis support system as an example of an embodiment;

FIG. 3 is a diagram for describing training phase processing by a first training processing unit and a second training processing unit of the image interpretation diagnosis support system as an example of an embodiment;

FIG. 4 is a diagram for describing a method of extracting a feature for each imaging protocol in the image interpretation diagnosis support system as an example of an embodiment;

FIG. 5 is a diagram for describing inference phase processing by a first inference processing unit and a second inference processing unit of the image interpretation diagnosis support system as an example of an embodiment;

FIG. 6 is a flowchart for describing the processing in the training phase in the image interpretation diagnosis support system as an example of an embodiment;

FIG. 7 is a diagram illustrating supervised data in the image interpretation diagnosis support system as an example of an embodiment;

FIG. 8 is a flowchart for describing the processing in the inference phase in the image interpretation diagnosis support system as an example of an embodiment; and

FIG. 9 is a diagram illustrating a diagnostic image data set to be inferred in the image interpretation diagnosis support system as an example of an embodiment.

### DESCRIPTION OF EMBODIMENTS

As described above, there are cases where the lesion can be easily found and cases where the pathological change can be less easily found in the medical images according to a type of a disease, depending on the imaging protocol. Therefore, in the existing image interpretation diagnosis using deep learning, the accuracy of inference (prediction) may decrease by using medical images with an imaging protocol that contributes little to the image interpretation diagnosis of a specific disease.

In one aspect, the embodiment aims to improve the accuracy of inference.

Hereinafter, embodiments according to the present image diagnosis support program, training program, information processing device, image diagnosis support method, and training method will be described with reference to the drawings. Note that the embodiments to be described below are merely examples, and there is no intention to exclude application of various modifications and techniques not explicitly described in the embodiments. For example, the present embodiments may be variously modified and implemented without departing from the gist thereof. Furthermore, each drawing is not intended to include only configuration elements illustrated in the drawing, and may include other functions and the like.

(A) Configuration

An image interpretation diagnosis support system 1 as one embodiment supports a doctor (image interpretation doctor) who interprets medical images, and presents a part having a high possibility of a lesion in the medical images and the degree of malignancy to the image interpretation doctor. For example, the present image interpretation diagnosis support system 1 infers (estimates) a part of a lesion from image data (diagnostic image data set) captured from a subject to be diagnosed, and outputs information indicating the estimated part of the lesion (lesion part). The subject includes a patient.

The image interpretation doctor, for example, diagnoses a patient based on the information presented by the image interpretation diagnosis support system 1.

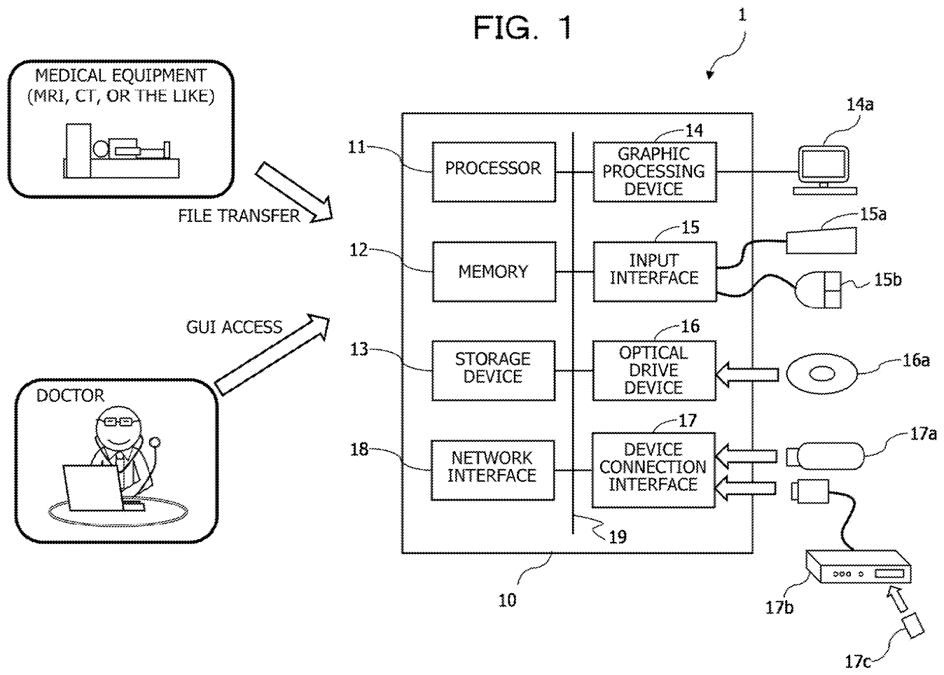

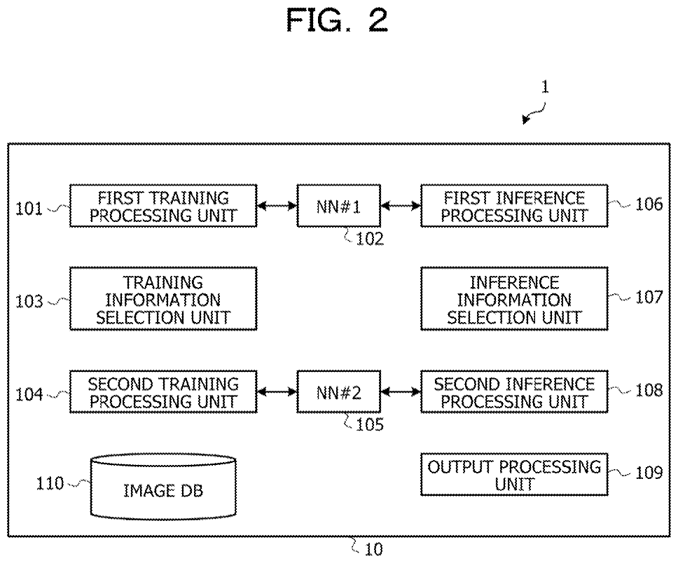

FIG. 1 is a diagram illustrating a hardware configuration of the image interpretation diagnosis support system 1 as an example of an embodiment, and FIG. 2 is a diagram illustrating a functional configuration thereof.

The image interpretation diagnosis support system 1 includes an inference server 10 as illustrated in FIG. 1. The inference server 10 receives input of medical images captured by medical equipment such as MRI and CT.

The medical equipment generates a plurality of types of medical images (image data) from the subject by switching imaging conditions (imaging protocols) and capturing (organs and affected parts of) the subject. The medical images captured by the medical equipment may be input to the inference server 10 by file transfer via a network (not illustrated), for example, and can be modified as appropriate.

The doctor accesses the inference server 10 through a graphical user interface (GUI) and performs various input operations, using an information processing device such as a personal computer (PC) connected via a network.

The inference server 10 is a computer (information processing device), and includes, for example, a processor 11, a memory 12, a storage device 13, a graphic processing device 14, an input interface 15, an optical drive device 16, a device connection interface 17, and a network interface 18 as configuration elements, as illustrated in FIG. 1. These configuration elements 11 to 18 are configured to be communicable with each other via a bus 19.

The processor (control unit) 11 controls the entire inference server 10. The processor 11 may be a multiprocessor. The processor 11 may also be, for example, any one of a central processing unit (CPU), a micro processing unit (MPU), a digital signal processor (DSP), an application specific integrated circuit (ASIC), a programmable logic device (PLD), a field programmable gate array (FPGA), and a graphics processing unit (GPU). Furthermore, the processor 11 may also be a combination of two or more types of elements of the CPU, MPU, DSP, ASIC, PLD, FPGA, and GPU.

Then, the processor 11 executes programs (an image diagnosis support program, a training program, and an operating system (OS) program) recorded in, for example, a computer-readable non-transitory recording medium, so that functions as a first training processing unit 101, a first neural network 102, a training information selection unit 103, a second training processing unit 104, a second neural network 105, a first inference processing unit 106, an inference information selection unit 107, a second inference processing unit 108, an output processing unit 109, and an image database 110, as illustrated in FIG. 2, are implemented.

The programs in which processing content to be executed by the inference server 10 is described can be recorded in various recording media. For example, the programs to be executed by the inference server 10 can be stored in the storage device 13. The processor 11 loads at least some of the programs in the storage device 13 to the memory 12 and executes the loaded programs.

Furthermore, the programs to be executed by the inference server 10 (processor 11) can also be recorded in a non-transitory portable recording medium such as an optical disk 16a, a memory device 17a, or a memory card 17c. The programs stored in the portable recording medium can be executed after being installed in the storage device 13, for example, under the control of the processor 11. Furthermore, the processor 11 may directly read the programs from the portable recording medium and execute the programs.

The memory 12 is a storage memory including a read only memory (ROM) and a random access memory (RAM). The RAM of the memory 12 is used as a main storage device of the inference server 10. The RAM temporarily stores at least some of the programs to be executed by the processor 11. Furthermore, the memory 12 stores various types of data needed for the processing by the processor 11.

The storage device 13 is a storage device such as a hard disk drive (HDD), a solid state drive (SSD), or a storage class memory (SCM) and stores various types of data. The storage device 13 is used as an auxiliary storage device of the inference server 10.

The storage device 13 stores the OS program, control programs, and various types of data. The control programs include the image diagnosis support program and the training program.

Note that a semiconductor storage device such as an SCM or a flash memory can also be used as the auxiliary storage device. Furthermore, redundant arrays of inexpensive disks (RAID) may be configured using a plurality of the storage devices 13.

The graphic processing device 14 is connected to a monitor 14a. The graphic processing device 14 displays an image on a screen of the monitor 14a in accordance with an instruction from the processor 11. Examples of the monitor 14a include a display device using a cathode ray tube (CRT) and a liquid crystal display device.

The input interface 15 is connected to a keyboard 15a and a mouse 15b. The input interface 15 transmits signals sent from the keyboard 15a and the mouse 15b to the processor 11. Note that the mouse 15b is an example of a pointing device, and other pointing devices can also be used. Examples of the other pointing devices include a touch panel, a tablet, a touch pad, and a track ball.

The optical drive device 16 reads data recorded in the optical disk 16a using laser light or the like. The optical disk 16a is a non-transitory portable recording medium having data recorded in a readable manner by reflection of light. Examples of the optical disk 16a include a digital versatile disc (DVD), a DVD-RAM, a compact disc read only memory (CD-ROM), and a CD-recordable (R)/rewritable (RW).

The device connection interface 17 is a communication interface for connecting peripheral devices to the inference server 10. For example, the device connection interface 17 can be connected to the memory device 17a or a memory reader/writer 17b. The memory device 17a is a non-transitory recording medium equipped with a communication function with the device connection interface 17, such as a universal serial bus (USB) memory. The memory reader/writer 17b writes data to the memory card 17c or reads data from the memory card 17c. The memory card 17c is a card-type non-transitory recording medium.

The network interface 18 is connected to the network. The network interface 18 transmits and receives data via the network. Other information processing devices, communication devices, and the like may be connected to the network. For example, the inference server 10 may be connected to the medical equipment via the network interface 18 and the network, and may receive the medical images from the medical equipment by file transfer.

Furthermore, the inference server 10 may also be connected to a database system that stores the medical images via the network interface 18 and the network, and receive the medical images from this database system.

The inference server 10 has the functions as the first training processing unit 101, the first neural network 102, the training information selection unit 103, the second training processing unit 104, the second neural network 105, the first inference processing unit 106, the inference information selection unit 107, the second inference processing unit 108, the output processing unit 109, and the image database 110, as illustrated in FIG. 2

In the image interpretation diagnosis support system 1, the functions as the first training processing unit 101, the training information selection unit 103, and the second training processing unit 104 are implemented by the processor 11 executing the training program. These first training processing unit 101, training information selection unit 103, and second training processing unit 104 function in a training phase.

Furthermore, the functions as the first inference processing unit 106, the inference information selection unit 107, the second inference processing unit 108, and the output processing unit 109 are implemented by the processor 11 executing the image diagnosis support program. These first inference processing unit 106, inference information selection unit 107, second inference processing unit 108, and output processing unit 109 function in an inference phase.

Furthermore, the image database 110 stores the medical images, supervised data, and the like to be used in the training phase and the inference phase.

The first training processing unit 101 performs training (machine learning) of the first neural network 102 in the training phase. The first neural network 102 may be denoted as NN #1.

The first neural network 102 implements a machine learning model (first model) that classifies a disease with which the subject is affected.

The first neural network 102 makes an inference by inputting a plurality of pieces of image data (diagnostic image data set) captured from one subject under a plurality of types of imaging conditions (imaging protocols), and outputs information of the name of a disease (an estimation result of the name of the subject's disease) with which the subject is inferred to be affected, and an absolute value of a feature for each imaging protocol calculated in the process of the inference.

The first training processing unit 101 generates the first neural network 102, using training data (supervised data) in which a first training image data set (a plurality of training images) respectively obtained by imaging the subject under mutually different imaging conditions (imaging protocols) is associated with the disease name (affected disease name information) of the subject.

Then, the first training processing unit 101 performs machine learning (training or first training) for the first neural network 102, using a first training data set (first training data) in which the first training image data set (a plurality of training images) respectively obtained by imaging the subject under mutually different imaging protocols (imaging conditions) is associated with the affected disease name information (disease name) of the subject.

The first neural network 102 may be a hardware circuit, or may be a virtual network by software that connects layers virtually constructed on a computer program by the processor 11. For the machine learning model, deep learning using a neural network with convolutional layers may be used, for example.

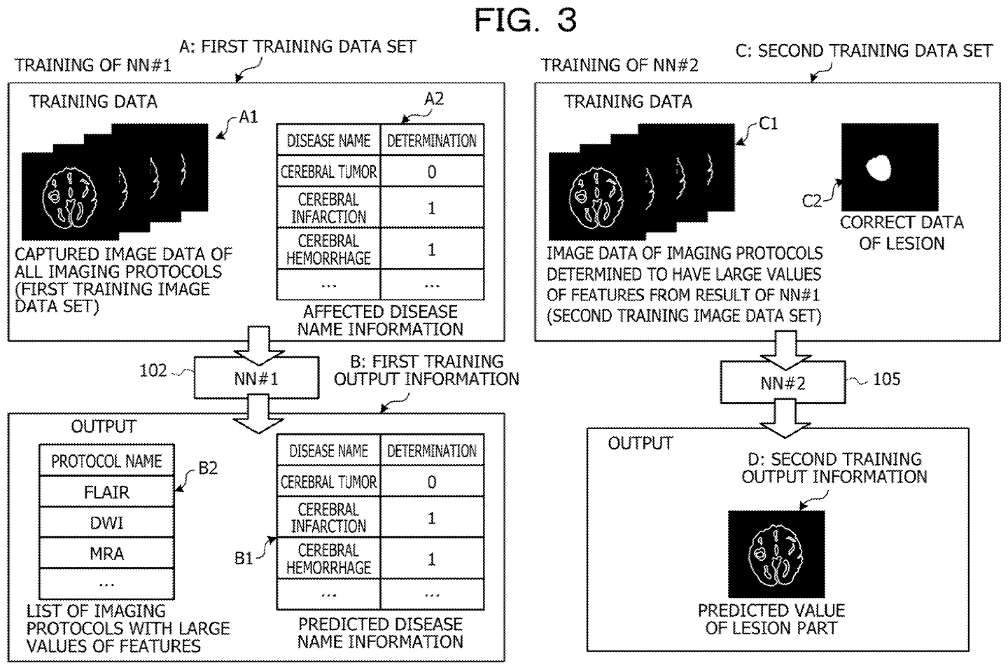

FIG. 3 is a diagram for describing training phase processing by the first training processing unit 101 and the second training processing unit 104 of the image interpretation diagnosis support system 1 as an example of an embodiment.

In FIG. 3, symbol A denotes the first training data set to be used for training the first neural network 102. The first training data set exemplified by symbol A is information generated from one subject, and includes input data (see symbol A1) and correct data (see symbol A2). In the present image interpretation diagnosis support system 1, the first training data set exemplified by symbol A is generated for each of a plurality of patients.

The input data included in the first training data set is a plurality of pieces of image data (medical images) obtained by capturing a patient with a plurality of types of imaging protocols.

For the first training data set, it is desirable to use image data captured with as many types of imaging protocols as possible for each subject who provides the medical images. Therefore, in the present image interpretation diagnosis support system 1, image data of all of imaging protocols obtained from a patient using medical equipment are used as the plurality of pieces of image data.

Hereinafter, a plurality of pieces of image data obtained by capturing one subject using a plurality of types of imaging protocols, which is included in the first training data set, may be referred to as a first training image data set. The first training image data set is used as the input data to the first neural network 102 in the training phase and is used to train this first neural network 102.

The first training processing unit 101 inputs the first training image data set to the first neural network 102 and creates the machine learning model (first neural network 102) that outputs inference results for this input data by machine learning.

Furthermore, the correct data included in the first training data set is the affected disease name information indicating the disease name of the disease with which the subject is actually affected. This affected disease name information indicates the disease name diagnosed by the doctor for the subject.

In FIG. 3, symbol B denotes first training output information output from the first neural network 102. The first training output information includes predicted disease name information (see symbol B1 in FIG. 3) and feature information calculated for each imaging protocol (see symbol B2 in FIG. 3).

The predicted disease name information is information indicating the name of the disease inferred from the image data of the subject and with which the subject is inferred to be affected.

When the first training image data set is input, the first neural network 102 infers the disease name from the first training image data set and outputs the predicted disease name information.

The first training processing unit 101 may optimize parameters and the like of the neural network by updating the parameters and the like in a direction of reducing a loss function that defines an error between the inference result (predicted disease name information) of the machine learning model (first neural network 102) for the training data and the correct data (affected disease name information), using, for example, a gradient descent method.

Furthermore, the first neural network 102 includes a plurality of convolutional layers, and extracts a feature for each imaging protocol from a plurality of pieces of image data with different imaging protocols, which constitutes the first training image data set.

The first neural network 102 includes at least a number of convolutional layers, the number corresponding to the number of imaging protocols in the first training image data set. Each convolutional layer calculates each feature of the image data of the corresponding imaging protocol.

The first neural network 102 outputs an absolute value of the calculated feature of the image data for each imaging protocol. Hereinafter, for convenience, the absolute value of the feature of the image data for each imaging protocol may be simply referred to as the feature for each imaging protocol.

The feature for each imaging protocol calculated by each convolutional layer is calculated in the process of estimating the disease name by the first neural network 102 described above.

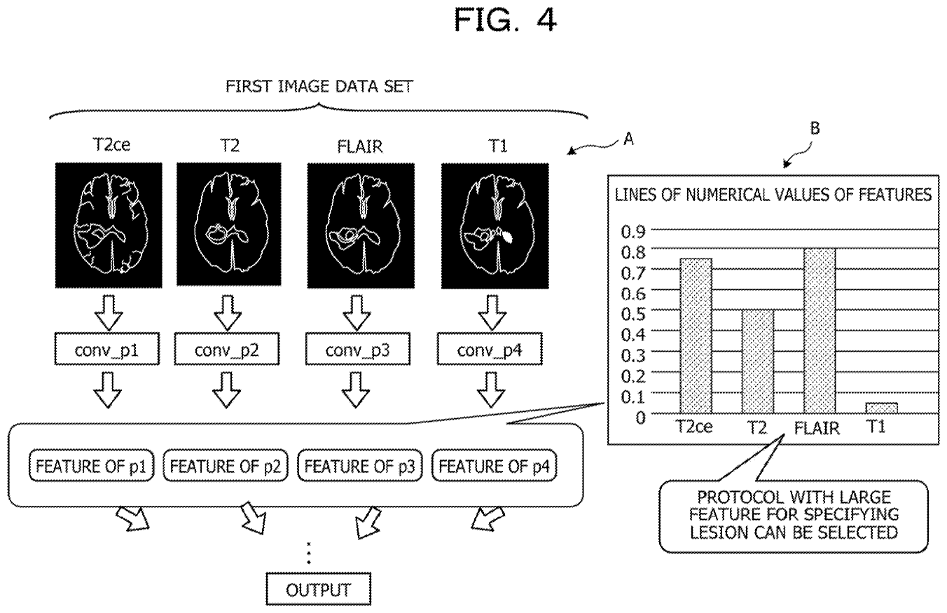

FIG. 4 is a diagram for describing a method of extracting a feature for each imaging protocol in the image interpretation diagnosis support system 1 as an example of an embodiment.

In the example illustrated in FIG. 4, the first training image data set including four pieces of image data captured with four types of imaging protocols T2ce, T2, FLAIR, and T1 is illustrated. Then, FIG. 4 illustrates a process of calculating the feature from each of pieces of the image data constituting the first training image data set (see symbol A in FIG. 4).

In FIG. 4, the first neural network 102 includes a convolutional layer conv_p1 provided corresponding to a feature protocol T2ce, a convolutional layer conv_p2 provided corresponding to a feature protocol T2, a convolutional layer conv_p3 provided corresponding to a feature protocol FLAIR, and a convolutional layer conv_p4 provided corresponding to a feature protocol T1.

In the first neural network 102, the convolutional layer conv_p1 calculates the feature (p1 feature) based on the image data of the feature protocol T2ce. Similarly, the convolutional layer conv_p2 calculates the feature (p2 feature) based on the image data of the feature protocol T2. Furthermore, the convolutional layer conv_p3 calculates the feature (p3 feature) based on the image data of the feature protocol FLAIR, and the convolutional layer conv_p4 calculates the feature (p4 feature) based on the image data of the feature protocol T1.

FIG. 4 illustrates a graph illustrating the feature for each imaging protocol calculated by each of the convolutional layers conv_p1 to conv_p4 (see symbol B in FIG. 4).

The first training processing unit 101 stores the feature for each imaging protocol, which has been calculated by each convolutional layer of the first neural network 102 based on the first training image data set, in a predetermined storage area such as the memory 12 or the storage device 13.

At this time, the first training processing unit 101 may sort the feature for each imaging protocol, which has been calculated by each convolutional layer, according to the value.

Symbol B2 in FIG. 3 denotes a display example of feature information calculated for each imaging protocol, and illustrates a list in which protocol names of the imaging protocols are arranged in descending order of the values of the features (information amounts).

The feature for each imaging protocol calculated by each of the convolutional layers conv_p1 to conv_p4 is used for disease name estimation by the first neural network 102 described above.

In the example illustrated in FIG. 3, the first neural network 102 outputs a list of the disease names of the diseases with which the patient is affected, and a list of the imaging protocols with large values of the features calculated in the process of inferring the disease names, using the image data of all the imaging protocols as input.

The feature for each imaging protocol corresponds to information that can specify how much the feature of the image by the corresponding imaging protocol has contributed to the inference. The imaging protocol with a large value of the feature indicates that the feature of the disease in the image data can be extracted in a large manner with the imaging protocol.

Note that the first neural network 102 may output the value of the feature calculated for each feature protocol.

The first neural network 102 corresponds to a first model that outputs the estimation result (predicted disease name information) of the disease name of the subject to be diagnosed and the degree of contribution (feature) to the estimation of each of the plurality of input images for each imaging condition according to the input of the plurality of input images obtained by imaging the subject to be diagnosed under different imaging conditions (imaging protocols).

The training information selection unit 103 selects training data to be used when the second training processing unit 104 trains the second neural network 105 from the first training image data set included in the first training data set. Hereinafter, one or more pieces of image data to be used for training the second neural network 105 will be referred to as a second training image data set.

The training information selection unit 103 determines the imaging protocol to be used for training (the imaging condition for training) based on the feature (the degree of contribution to the estimation) of each of the plurality of training images for each imaging protocol (imaging condition), which has been output by the first neural network 102 according to the input of the first training image data set (the plurality of training images).

Then, the training information selection unit 103 selects a second training image data set (training input image) imaged with the imaging protocol (the imaging condition for training) selected based on the feature from among the first training image data set (the plurality of training images).

For example, the training information selection unit 103 excludes, for example, the image data of the imaging protocol whose feature is less than a predetermined threshold from the first training image data set based on the feature for each imaging protocol calculated by the first neural network 102, and sets only the image data whose feature is equal to or larger than the predetermined threshold as the second training image data set.

By excluding the image data of the imaging protocol whose feature is less than a predetermined threshold from the second training image data set, use of the image data of the imaging protocol with a low feature (for example, with a small contribution to image interpretation diagnosis) in the training of the second neural network 105 is suppressed.

Furthermore, the training information selection unit 103 may sort the imaging protocols according to the values of the features, select a predetermined number of imaging protocols in descending order of the values of the features, select the image data captured with these imaging protocols from the first training image data set, and set the selected image data as the second training image data set.

The training information selection unit 103 stores the created second training image data set in a predetermined storage area such as the memory 12 or the storage device 13.

The second training processing unit 104 performs training (machine learning) of the second neural network 105 in the training phase. The second neural network 105 may be denoted as NN #2.

The second neural network 105 implements a machine learning model (second model) that infers a part of a lesion from the medical image data captured from the subject. The second neural network 105 may be a hardware circuit, or may be a virtual network by software connecting between layers virtually constructed on a computer program by the processor 11. For the machine learning model, deep learning using a neural network may be used, for example.

In FIG. 3, symbol C denotes the second training data set to be used for training a second neural network 105. The second training data set exemplified by symbol C is information generated from one subject, and includes input data (see symbol C1 in FIG. 3) and correct data (see symbol C2 in FIG. 3). In the present image interpretation diagnosis support system 1, the second training data set exemplified by symbol C is generated for each of a plurality of subjects.

The input data included in the second training data set is the second training image data set selected by the training information selection unit 103 (see symbol C1 in FIG. 3).

The second training image data set is used as the input data to the second neural network 105 in the training phase and is used to train this second neural network 105.

The second training processing unit 104 inputs the second training image data set to the second neural network 105 and creates the machine learning model (second neural network 105) that outputs inference results for this input data by machine learning.

Then, the second training processing unit 104 performs machine learning (training or second training) for the second neural network (second model) 105, using the second training data set (second training data) in which the second training image data set (training input image) is associated with the lesion part (correct data of the lesion).

Furthermore, the correct data included in the second training data set is lesion location information indicating a location of the lesion of the disease that the subject is actually affected with (see symbol C2 in FIG. 3). This lesion location information indicates the lesion part previously specified by the doctor in the medical image data captured from the subject.

In FIG. 3, symbol D denotes second training output information output from the second neural network 105.

When the second training image data set is input, the second neural network 105 infers the part of the lesion from the second training image data set, and outputs information (output image data) indicating a predicted part of the lesion.

In FIG. 3, the second training output information exemplified by symbol D indicates output image data visualized by reflecting a predicted value of the lesion part on the medical image.

The information of the part of the lesion inferred by the second neural network 105 may be referred to as predicted lesion part information.

The second training processing unit 104 causes the second neural network 105 to specify the lesion part.

The second training processing unit 104 checks an error (gap) between the lesion part specified by the second neural network 105 and the correct answer, and updates parameters of the second neural network 105 so that this error becomes smaller.

The second training processing unit 104 improves the accuracy of the second neural network 105 by repeatedly performing such specification of the lesion part by the second neural network 105 and update of the parameters of the second neural network 105 based on the gap between a specified result and the correct answer.

The second training processing unit 104 stores the created predicted lesion part information in a predetermined storage area such as the memory 12 or the storage device 13.

In the inference phase, the first inference processing unit 106 inputs a plurality of pieces of image data (diagnostic image data set) captured from the patient (subject) to be diagnosed to the first neural network 102, and obtains the inference result output by the first neural network 102.

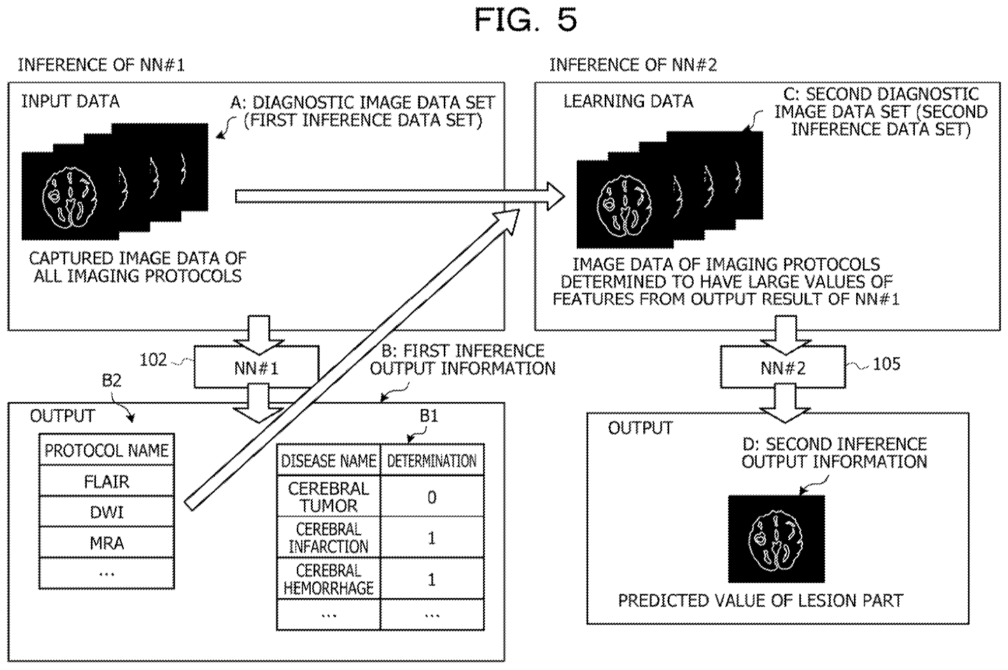

FIG. 5 is a diagram for describing inference phase processing by the first inference processing unit 106 and the second inference processing unit 108 of the image interpretation diagnosis support system 1 as an example of an embodiment.

In FIG. 5, symbol A denotes a diagnostic image data set input to the first neural network 102. The diagnostic image data set exemplified by symbol A is information generated from one patient to be diagnosed, and includes a plurality of pieces of image data created by capturing the patient with a plurality of types of imaging protocols. These pieces of image data are medical images captured by medical equipment such as MRI and CT.

The plurality of pieces of image data constituting the diagnostic image data set corresponds to inference target data that is data to be estimated of a lesion in the image interpretation diagnosis support system 1. The diagnostic image data set is a plurality of pieces of image data in which lesions and the like are unknown. The diagnostic image data set may be referred to as input data.

For the diagnostic image data set, it is desirable to use image data of the patient captured with as many types of imaging protocols as possible. Therefore, in the present image interpretation diagnosis support system 1, it is desirable to use the image data of all the imaging protocols that can be acquired from the patient in the present image interpretation diagnosis support system 1 as the diagnostic image data set.

The first inference processing unit 106 inputs the diagnostic image data set to the first neural network 102 and acquires the inference result for this input data (diagnostic image data set). The diagnostic image data set may be referred to as a first inference data set.

In FIG. 5, symbol B denotes first inference output information output from the first neural network 102 in the inference phase.

The first inference output information includes predicted disease name information (see symbol B1 in FIG. 5) and feature information calculated for each imaging protocol (see symbol B2 in FIG. 5).

When the diagnostic image data set is input, the first neural network 102 infers the disease name from the diagnostic image data set and outputs it.

In the predicted disease name information exemplified by symbol B1 in FIG. 5, the disease name is associated with a determination result as to whether the patient is affected with the disease. For example, “1” is set in the determination for cerebral infarction and cerebral hemorrhage, indicating that the patient has been inferred to be affected with cerebral infarction and cerebral hemorrhage.

Furthermore, the first neural network 102 extracts (calculates) an absolute value of the feature for each imaging protocol from the plurality of pieces of image data with different imaging protocols, which constitutes the diagnostic image data set, in the inference phase.

The first inference processing unit 106 stores the feature for each imaging protocol, which has been calculated by each convolutional layer of the first neural network 102 based on the first training image data set, in a predetermined storage area such as the memory 12 or the storage device 13, in the inference process.

At this time, the first inference processing unit 106 may sort the feature for each imaging protocol, which has been calculated by each convolutional layer, according to each value.

Symbol B2 in FIG. 5 illustrates a list in which protocol names of the imaging protocols are arranged in descending order of the values of the features (information amounts).

Each feature calculated for each imaging protocol represents how much the feature of the image data captured with the corresponding imaging protocol has contributed to the inference.

The inference information selection unit 107 selects inference data (input data) that the second inference processing unit 108 inputs to the second neural network 105 from the above-described diagnostic image data set based on the feature for each imaging protocol calculated by the first inference processing unit 106. Hereinafter, one or more image data input to the second neural network 105 in the inference phase will be referred to as a second diagnostic image data set.

The inference information selection unit 107 selects the imaging protocol to be used for inference from among the plurality of types of imaging protocols used to capture the plurality of pieces of image data that constitutes the diagnostic image data set based on the feature for each imaging protocol calculated by the first inference processing unit 106.

For example, the inference information selection unit 107 selects input images (second diagnostic image data set) imaged under the imaging condition for estimation (the imaging protocol to be used for inference) selected based on the feature (the degree of contribution) from among the plurality of input images (diagnostic image data set) to be determined. The second diagnostic image data set may be referred to as a second inference data set.

In FIG. 5, symbol C denotes the second diagnostic image data set selected by the inference information selection unit 107.

The second diagnostic image data set is a set of image data of the imaging protocols that are determined to have large values of the features selected based on the feature output from the first neural network 102. The second diagnostic image data set corresponds to an input image imaged with the imaging protocol (imaging condition for estimation) to be used for inference.

The inference information selection unit 107 preferentially selects the imaging protocol with a large value of the feature based on the feature for each imaging protocol. For example, the imaging protocols whose feature is less than a predetermined threshold may be excluded, and only the imaging protocols whose feature is equal to or greater than the threshold may be selected. Furthermore, the inference information selection unit 107 may select a predetermined number of imaging protocols that are higher in descending order of the values of the features.

The inference information selection unit 107 selects the image data captured with the imaging protocols selected as described above from the diagnostic image data set and creates the second diagnostic image data set.

The inference information selection unit 107 excludes the image data of the imaging protocol whose feature is less than a predetermined threshold, selects only the image data of the imaging protocol whose feature is equal to or greater than the threshold, and creates the second diagnostic image data set. Therefore, the use of the image data of the imaging protocol with a low value of the feature (for example, with a small contribution to image interpretation diagnosis) is suppressed in the inference by the second neural network 105.

The inference information selection unit 107 stores the created second diagnostic image data set in a predetermined storage area such as the memory 12 or the storage device 13.

The second inference processing unit 108 makes an inference using the second neural network 105 in the inference phase. The second inference processing unit 108 inputs the second diagnostic image data set selected by the inference information selection unit 107 to the second neural network 105 and obtains the inference result output by the second neural network 105.

The second inference processing unit 108 inputs the second inference data set (input images) imaged under the imaging condition for estimation (the imaging protocol to be used for inference) to the second neural network 105 (second model) that outputs the estimation result of the lesion part in the input image according to the input image.

When the second diagnostic image data set is input, the second neural network 105 infers the part of the lesion from the second diagnostic image data set, and outputs information indicating the predicted part of the lesion (the predicted value of the lesion part).

In FIG. 5, symbol D denotes second inference output information that is the inference result output by the second neural network 105. The second inference output information exemplified by symbol D includes output image data visualized by reflecting a predicted value of the lesion part on the medical image.

In the inference phase, the information of the part of the lesion inferred by the second neural network 105 may be referred to as predicted lesion part information.

The second inference processing unit 108 stores the created predicted lesion part information in a predetermined storage area such as the memory 12 or the storage device 13.

The output processing unit 109 performs processing of outputting the second inference output information output by the second neural network 105 to an outside of the inference server 10. For example, the output processing unit 109 may perform processing of transferring the second inference output information to a PC used by the doctor, which is connected to the inference server 10 via a network. Furthermore, the output processing unit 109 may display the second inference output information on the monitor 14a and may further store the second inference output information in the memory card 17c, and appropriate modifications may be made and implemented.

The output processing unit 109 outputs the estimation result of the lesion part specified based on the output result of the second neural network 105 (second model).

The image database 110 stores medical images. For example, the image database 110 stores various types of image data constituting the first training data set, the affected disease name information, and the like.

(B) Operation

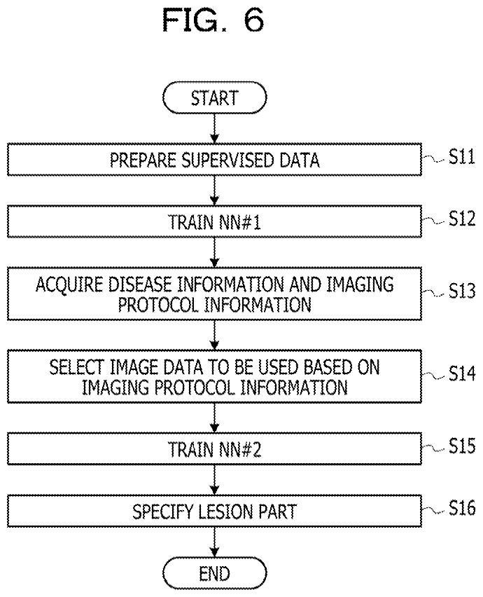

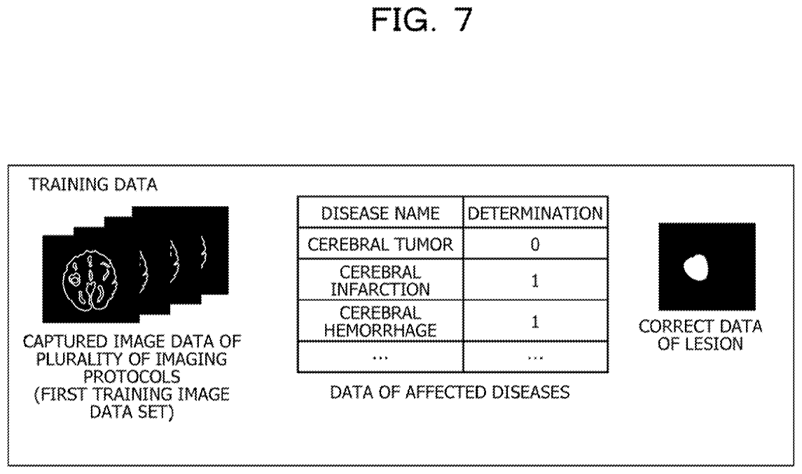

The processing in the training phase in the image interpretation diagnosis support system 1 as an example of the embodiment configured as described above will be described with reference to FIG. 7 according to the flowchart (steps S11 to S16) illustrated in FIG. 6. FIG. 7 is a diagram illustrating supervised data in the image interpretation diagnosis support system 1 as an example of an embodiment.

In step S11, the first training processing unit 101 prepares supervised data.

FIG. 7 illustrates supervised data to be used for one-time training in the training phase, and illustrates the supervised data created based on one subject.

For example, the supervised data for one subject includes a plurality of pieces of medical image data (first training image data set) obtained by capturing the subject with a plurality of types of imaging protocols, the affected disease name information indicating the disease name of the disease with which the subject is actually affected, and the correct data (mask image data) of the lesion part for each disease.

he first training processing unit 101 may read each piece of data constituting the supervised data from the image database 110.

In step S12, the first training processing unit 101 inputs the first training image data set to the first neural network 102, and trains the first neural network 102 using the affected disease name information as the correct data.

In step S13, the training information selection unit 103 acquires the feature (protocol information) for each imaging protocol calculated by the first neural network 102 and the inferred predicted disease name information (disease information).

In step S14, the training information selection unit 103 selects the second training image data set to be used when the second training processing unit 104 trains the second neural network 105 from the first training image data set based on the feature (protocol information) for each imaging protocol.

For example, the training information selection unit 103 selects the image data whose feature is equal to or greater than a predetermined threshold from one training image data set as the second training image data set.

In step S15, the second training processing unit 104 inputs the second training image data set to the second neural network 105, and trains the second neural network 105 using the correct data of the lesion part for each disease as correct data.

In step S16, the second training processing unit 104 causes the second neural network 105 to specify the lesion part.

The second training processing unit 104 checks an error between the lesion part specified by the second neural network 105 and the correct answer, and updates parameters of the second neural network 105 so that this error becomes smaller. Thereafter, the processing ends.

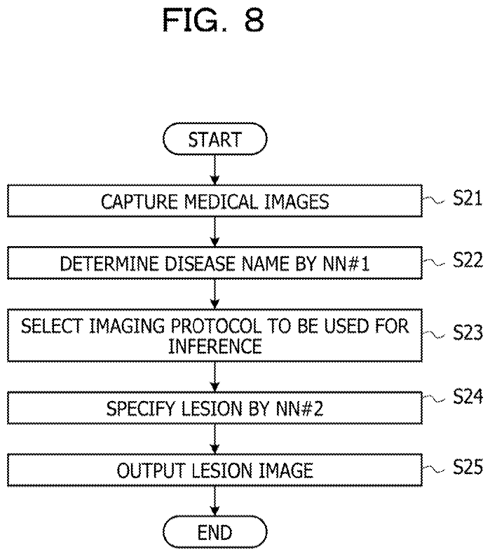

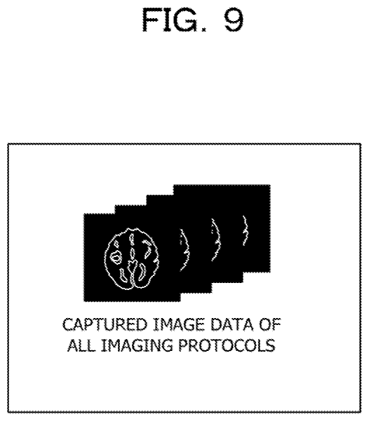

Next, the processing in the inference phase in the image interpretation diagnosis support system 1 as an example of the embodiment configured as described above will be described with reference to FIG. 9 according to the flowchart (steps S21 to S25) illustrated in FIG. 8. FIG. 9 is a diagram illustrating the diagnostic image data set to be inferred in the image interpretation diagnosis support system 1 as an example of an embodiment.

In step S21, the medical equipment creates a plurality of pieces of image data (medical images) by capturing the patient with a plurality of types of imaging protocols.

FIG. 9 illustrates the diagnostic image data set to be inferred in the inference phase. In the present image interpretation diagnosis support system 1, the diagnostic image data set is image data captured from a patient whose lesion and the like are unknown, and is created by capturing one patient with a plurality of types of imaging protocols using medical equipment.

In step S22, the first inference processing unit 106 inputs the diagnostic image data set to the first neural network 102, and the first inference output information (the predicted disease name information, the feature information calculated for each imaging protocol) is output. For example, the first neural network 102 diagnoses the disease name.

In step S23, the inference information selection unit 107 selects the imaging protocol to be used for inference from among the plurality of types of imaging protocols used to capture the plurality of pieces of image data that constitutes the diagnostic image data set based on the feature for each imaging protocol calculated by the first inference processing unit 106.

At this time, the inference information selection unit 107 selects the imaging protocol with a large value of the feature (for example, the imaging protocol whose value of the feature is equal to or greater than a threshold or the imaging protocol with a high value of the feature).

Then, the inference information selection unit 107 selects the image data captured with the imaging protocols selected from the diagnostic image data set and creates the second diagnostic image data set.

In step S24, the second inference processing unit 108 inputs the second diagnostic image data set selected by the inference information selection unit 107 to the second neural network 105. When the second diagnostic image data is input, the second neural network 105 infers the part of the lesion from the second diagnostic image data, and outputs the second inference output information.

In step S25, the output processing unit 109 transfers (outputs) the second inference output information (lesion image) output from the second neural network 105 to the PC used by the doctor. Thereafter, the processing ends.

(C) Effects

As described above, according to the image interpretation diagnosis support system 1 as an example of an embodiment, the inference information selection unit 107 excludes the image data of the imaging protocol whose feature is less than a predetermined threshold from the second diagnostic image data set, for example, selects the image data of the imaging protocol whose feature is equal to or greater than the threshold, and creates the second diagnostic image data set, in the inference phase.

Then, when the second inference processing unit 108 inputs the second diagnostic image data set to the second neural network 105, the second neural network 105 infers the part of the lesion from the second diagnostic image data set.

Therefore, the use of the image data of the imaging protocol with a low feature (for example, with a small contribution to image interpretation diagnosis) is suppressed in the inference by the second neural network 105. Therefore, in the inference by the second neural network 105, only the image data of the imaging protocol with high contribution to the image interpretation diagnosis of the disease is used, so the inference accuracy by the second neural network 105 can be improved.

For example, the inference information selection unit 107 may select the image data of a predetermined number of imaging protocols in descending order of the values of the features and create the second diagnostic image data set. This also suppresses the use of the image data of the imaging protocol with a low feature (for example, with a small contribution to image interpretation diagnosis), and improves the inference accuracy of the second neural network 105.

Furthermore, in the training phase, the training information selection unit 103 excludes the image data of the imaging protocol whose feature is less than a predetermined threshold from the second training image data set, selects the image data of the imaging protocol whose feature is equal to or greater than the threshold, and creates the second training image data set.

Then, when the second training processing unit 104 inputs the second training image data set to the second neural network 105, the second neural network 105 infers the part of the lesion from the second training image data set.

Therefore, the use of the image data of the imaging protocol with a low feature (for example, with a small contribution to image interpretation diagnosis) is suppressed in the training of the second neural network 105. Therefore, in the training of the second neural network 105, only the image data of the imaging protocol with a high contribution to the image interpretation diagnosis of the disease is used, so the accuracy of the second neural network 105 can be improved.

Furthermore, the training information selection unit 103 may sort the imaging protocols according to the values of the features, select a predetermined number of imaging protocols in descending order of the values of the features, select the image data captured with these imaging protocols from the first training image data set, and set the selected image data as the second training image data set. This also suppresses the use of the image data of the imaging protocol with a low feature (for example, with a small contribution to image interpretation diagnosis), and improves the inference accuracy of the second neural network 105.

(D) Others

Each configuration and each processing of the present embodiment may be selected or omitted as needed or may be appropriately combined.

Then, the disclosed technique is not limited to the embodiment described above, and various modifications may be made and carried out without departing from the gist of the present embodiment.

For example, the functions of the inference information selection unit 107 and the training information selection unit 103 may be provided in the first neural network 102 (NN #1).

Furthermore, the present embodiment may be implemented and manufactured by those skilled in the art according to the disclosure described above.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A non-transitory computer-readable storage medium storing an image diagnosis support program for causing a computer to execute a process, the process comprising:
inputting a plurality of input images to a first model that is generated by using training data in which a plurality of training images obtained by imaging a first subject under a plurality of imaging conditions is associated with a disease name of the first subject, the first model outputting, according to input of a plurality of input images obtained by imaging a second subject under the plurality of imaging conditions, an estimation result of a disease name of the second subject, and a degree of contribution to estimation of each of the plurality of input images for each of the imaging conditions;
selecting, among the plurality of input images, an input image imaged under an imaging condition for estimation selected based on the degree of contribution;
inputting the input image imaged under the imaging condition for estimation to a second model that outputs an estimation result of a lesion part in the input image according to the input of the input image; and
outputting the estimation result of the lesion part specified based on an output result of the second model.

2. The non-transitory computer-readable storage medium according to claim 1, wherein the process further comprising
excluding an imaging condition whose degree of contribution is less than a certain threshold from the imaging condition for estimation.

3. The non-transitory computer-readable storage medium according to claim 1, wherein the process further comprising adding an imaging condition that is highest in the degree of contribution to the imaging condition for estimation.

4. An information processing device comprising:
one or more memories; and
one or more processors coupled to the one or more memories and the one or more processors configured to:
input a plurality of input images to a first model that is generated by using training data in which a plurality of training images obtained by imaging a first subject under a plurality of imaging conditions is associated with a disease name of the first subject, the first model outputting, according to input of a plurality of input images obtained by imaging a second subject under the plurality of imaging conditions, an estimation result of a disease name of the second subject, and a degree of contribution to estimation of each of the plurality of input images for each of the imaging conditions,
select, among the plurality of input images, an input image imaged under an imaging condition for estimation selected based on the degree of contribution,
input the input image imaged under the imaging condition for estimation to a second model that outputs an estimation result of a lesion part in the input image according to the input of the input image, and
output the estimation result of the lesion part specified based on an output result of the second model.

5. The information processing device according to claim 4, wherein the one or more processors are further configured to exclude an imaging condition whose degree of contribution is less than a certain threshold from the imaging condition for estimation.

6. The information processing device according to claim 4, wherein the one or more processors are further configured to
add an imaging condition that is highest in the degree of contribution to the imaging condition for estimation.

7. An image diagnosis support method for a computer to execute a process comprising:
inputting a plurality of input images to a first model that is generated by using training data in which a plurality of training images obtained by imaging a first subject under a plurality of imaging conditions is associated with a disease name of the first subject, the first model outputting, according to input of a plurality of input images obtained by imaging a second subject under the plurality of imaging conditions, an estimation result of a disease name of the second subject, and a degree of contribution to estimation of each of the plurality of input images for each of the imaging conditions;
selecting, among the plurality of input images, an input image imaged under an imaging condition for estimation selected based on the degree of contribution;
inputting the input image imaged under the imaging condition for estimation to a second model that outputs an estimation result of a lesion part in the input image according to the input of the input image; and
outputting the estimation result of the lesion part specified based on an output result of the second model.

8. The image diagnosis support method according to claim 7, wherein the process further comprising
excluding an imaging condition whose degree of contribution is less than a certain threshold from the imaging condition for estimation.

9. The image diagnosis support method according to claim 7, wherein the process further comprising
adding an imaging condition that is highest in the degree of contribution to the imaging condition for estimation.
